(19) United States
(12) Patent Application Publication
(10) Pub. No.: US 2019/0021993 A1
(43) Pub. Date: Jan. 24, 2019

(54) Breathing Strip Aid for Mask

(71) Applicant: Leo GREEN, Rienzi, MS (US)

(72) Inventor: Leo GREEN, Rienzi, MS (US)

(21) Appl. No.: 15/654,067

(22) Filed: Jul. 19, 2017

(51) Int. Cl. A61K 9/00; A61M 16/06; A41D 13/11

(52) U.S. Cl. CPC A61K 9/0073; A62B 23/06; A41D 13/1192; A61M 16/0666

(57) ABSTRACT

An antibacterial breathing strip for nasal decongestant and cough suppressant includes a flexible sheet and an enclosing panel that is traversed with a plurality of ventilation openings. The flexible sheet and the enclosing panel are perimetrically connected to each other while enveloping a filter pad that is saturated with a quantity of antibiotic ointment. The antibacterial breathing strip is intended to externally couple with a mask so that when an individual breathes into the mask the user aromatically inhales the ointment properties, which is controlled and released from the breathing strip via designated ventilation openings, to promote nasal decongestant, suppressing coughs, the removing of sinus pressure, and the removing of sinus headaches and bronchitis.

### FIELD OF THE INVENTION

The present invention relates generally to a breathing strip. More specifically, the present invention is an antibacterial breathing strip for nasal decongestant and cough suppressant that purifies breathing air as the present invention is configured to be adhered to a mask.

### BACKGROUND OF THE INVENTION

Individuals normally wear protective masks during many different practical events as each mask is distinct from one another upon their functionality. For example, medical personals utilize protective masks as oxygen masks, anesthetic masks, burn masks, and surgical masks. Athletes utilize protective masks during different sporting event such as football, hockey, and fencing. Due to many air pollution and bacteria that circulate through air, breathing air has become unhealthy for many people. As a result, many individuals desire to wear protective masks so that they are able to breath clean and purified air. Even though there are many different masks that are able to purify breathing air, they are unable to perform as antibacterial filters.

It is therefore an objective of the present invention to provide an antibacterial breathing strip for nasal decongestant and cough suppressant, wherein the present invention is designed to adhered or couple with a surgical mask or any other similar types of flexible masks. More specifically, a filter pad that is saturated with a quantity of antibiotic ointment is enclosed with a flexible sheet so that the present invention is externally able to couple a flexible mask. As a result, the present invention provides an inexpensive and effective solution to purify and sterile polluted breathing air.

### BRIEF DESCRIPTION OF THE DRAWINGS

FIG. 1 is a front perspective view of the present invention coupled onto the mask.

FIG. 2 is a front perspective view of the present invention.

FIG. 3 is a rear perspective view of the present invention.

FIG. 4 is a top view of the present invention, showing the plane upon which a cross sectional view is taken shown in FIG. 5.

FIG. 5 is a cross section view of the present invention taken along line A-A of FIG. 4, showing the filter pad.

FIG. 6 is a schematic view showing the filter pad of the present invention.

FIG. 7 is a schematic view showing the ingredients of the antibiotic ointment of the present invention.

### DETAIL DESCRIPTIONS OF THE INVENTION

All illustrations of the drawings are for the purpose of describing selected versions of the present invention and are not intended to limit the scope of the present invention.

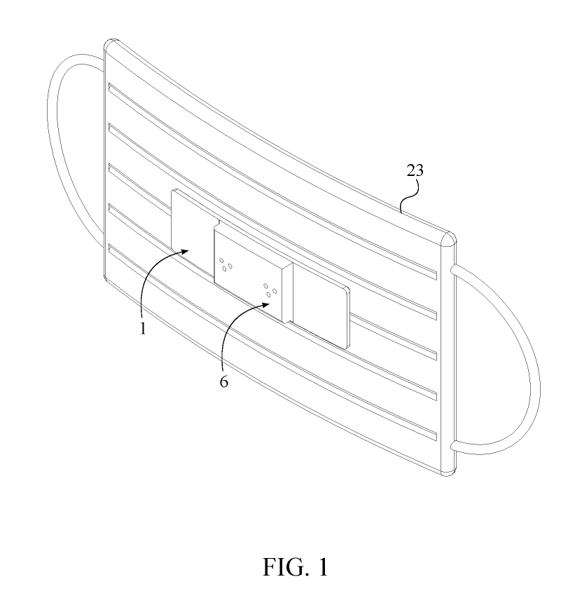

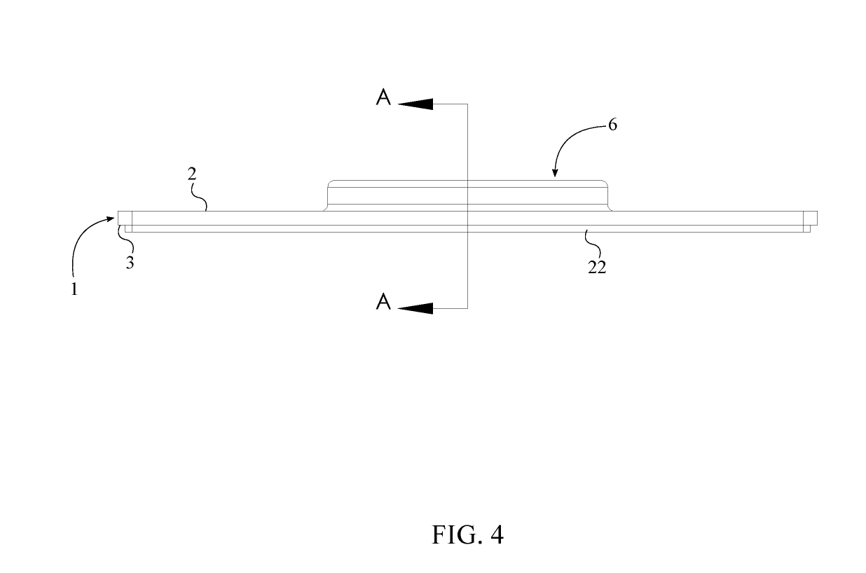

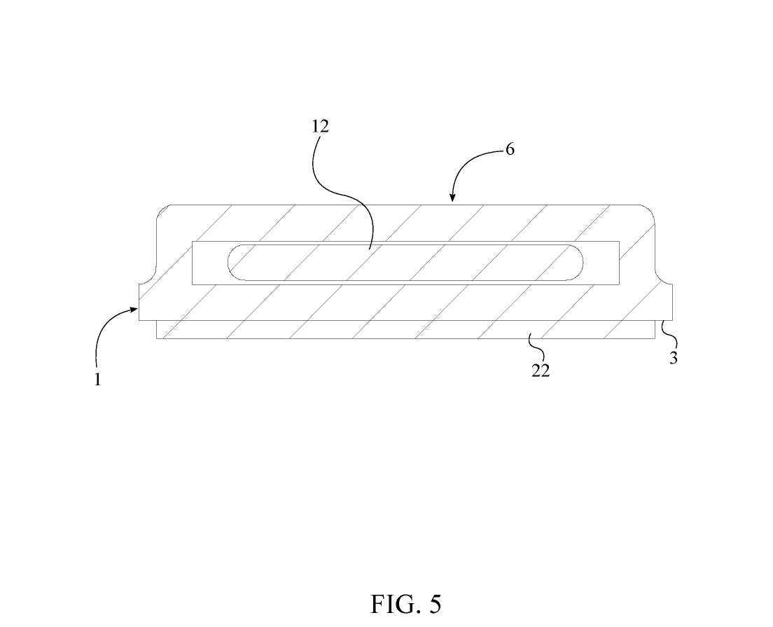

The present invention is an antibacterial breathing strip for nasal decongestant and cough suppressant as the present invention purifies breathing air. The present invention is coupled to a mask 23 so that the mask 23 can be positioned adjacent to a face of an individual covering the nostril openings. More specifically, the present invention is coupled to the mask 23 with a fastening mechanism 21 as the mask 23 can be any types of protective covers and the fastening mechanism 21 can be any types of fasteners that couples the present invention to the mask 23. The present invention comprises a flexible sheet 1, an enclosing panel 6, a plurality of ventilation openings 11, a filter pad 12 in addition to the fastening mechanism 21 and the mask 23 as shown in FIG. 1-5. In reference to the general configuration of the present invention, the plurality of ventilation openings 11 traverses through the enclosing panel 6. The plurality of ventilation openings 11 provides a pathway for breathing air to enter so that the present invention can initiate the air purification process of breathing air as an individual inhale. The filter pad 12 that functions as the antibacterial member is positioned in between the flexible sheet 1 and the enclosing panel 6 as the enclosing panel 6 is perimetrically connected onto the flexible sheet 1. The fastening mechanism 21 is coupled onto the flexible sheet 1 and positioned opposite of the enclosing panel 6 so that the present invention can be attached to the mask 23 through the fastening mechanism 21. Due to the configuration of the components, breathing air travels through the plurality of ventilation openings 11, the filter pad 12, and the flexible sheet 1, completing the air purification process as an individual inhale.

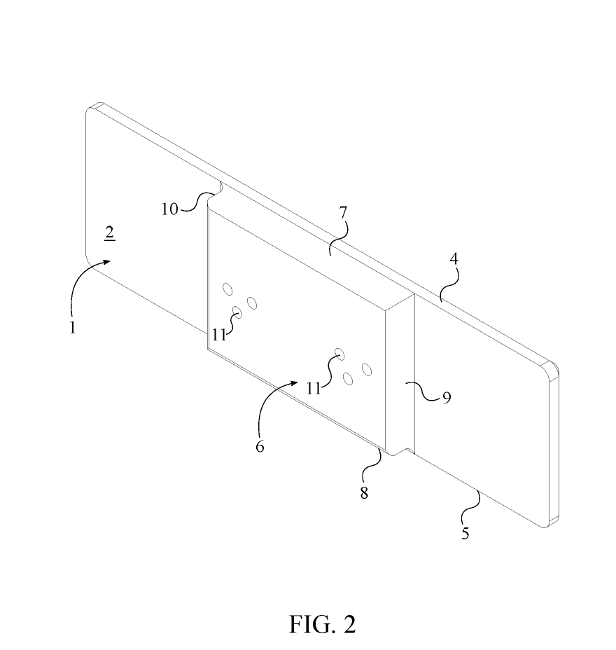

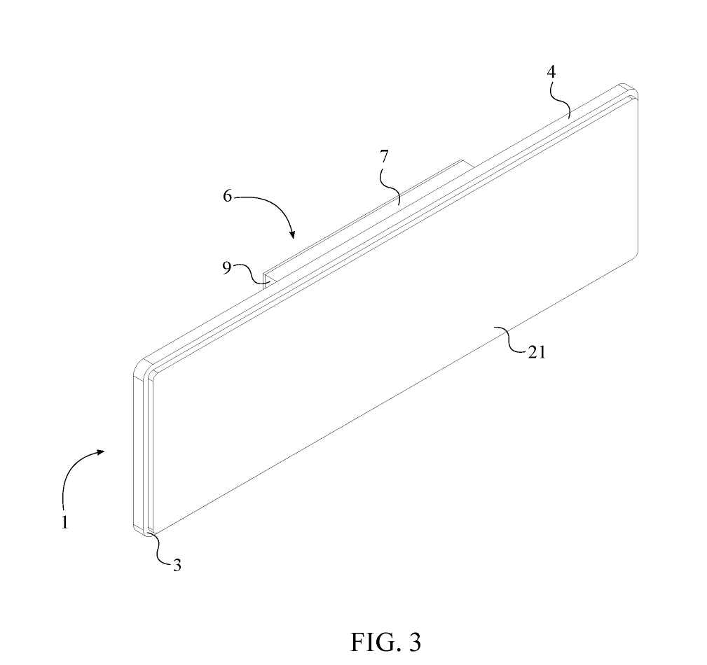

In reference to FIG. 2-3, the flexible sheet 1 is a generally made from woven fabric, plastic, or latex as manufactured material allows the present invention to deform according the shapes of the mask 23. The flexible sheet 1 is preferably formed into a rectangular shape so that the flexible sheet 1 is able to fully cover the nostril openings. However, the shape of the flexible sheet 1 is not limited to the rectangular shape and can be any other types of geometric shapes or organic shapes as long as the flexible sheet 1 is able to fully cover the nostril openings. Additionally, the flexible sheet 1 is also a permeable sheet so that breathing air can pass through after the filter pad 12 completes the air purification process.

In reference to FIG. 2-3, the enclosing panel 6 comprises a proximal edge 7, a distal edge 8, a first lateral edge 9, and a second lateral edge 10. The first lateral edge 9 and the second lateral edge 10 are extended in between the proximal edge 7 and the distal edge 8, delineating a rectangular shape for the enclosing panel 6. In reference to connection points between the flexible sheet 1 and the enclosing panel 6, the proximal edge 7 is terminally connected to a top edge 4 of the flexible sheet 1 while the distal edge 8 is terminally connected to a bottom edge 5 of the flexible sheet 1. Then, the first lateral edge 9 and the second lateral edge 10 are connected onto a front surface 2 of the flexible sheet 1 fully enclosing the filter pad 12 with the flexible sheet 1 and the enclosing panel 6. Simultaneously, the first lateral edge 9 and the second lateral edge 10 allow the flexible sheet 1 to extended beyond the enclosing panel 6. The extended sections of the flexible sheet 1 provide additional surface area to the present invention so that the fastening mechanism 21 can securely couple the present invention to the mask 23.

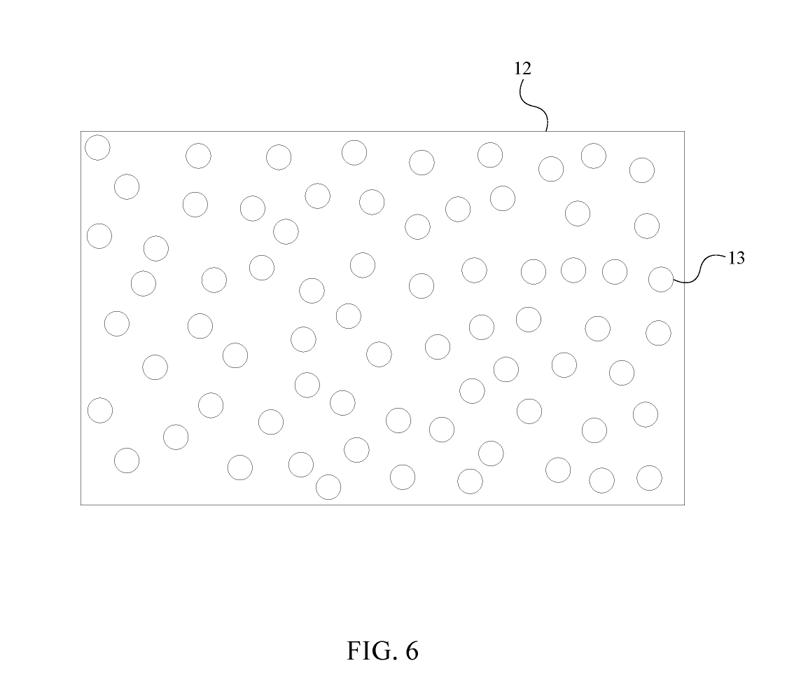

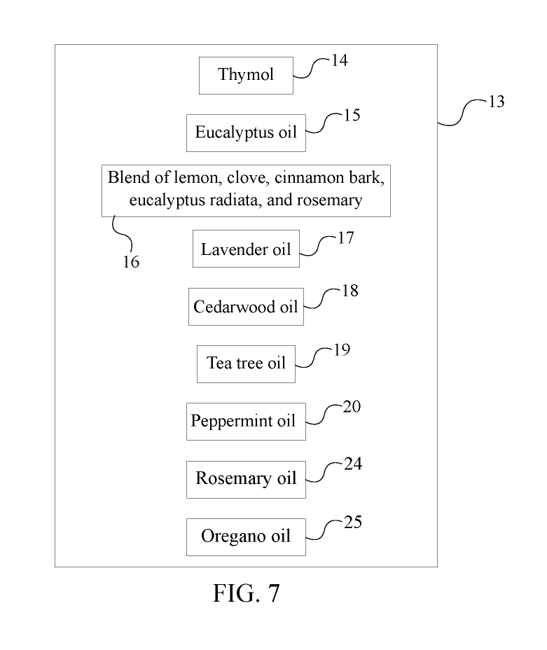

In reference to FIG. 6, the filter pad 12 is able to function as the antibacterial member due to a quantity of antibiotic ointment 13. More specifically, the filter pad 12 that is preferably made from cotton material is saturated with the quantity of antibiotic ointment 13. In reference to FIG. 7, the quantity of antibiotic ointment 13 comprises ingredient selected from the group consisting of a quantity of thymol 14, a quantity of eucalyptus oil 15, a blend of lemon, clove, cinnamon bark, eucalyptus radiata, and rosemary 16, a quantity of lavender oil 17, a quantity of cedarwood oil 18, a quantity of tea tree oil 19, a quantity of peppermint oil 20, a quantity of rosemary oil 24, a quantity of oregano oil 25, and combinations thereof. As a result of the abovementioned composition, the quantity of antibiotic ointment 13 is able to effectively purify any kind of airborne bacteria and pollutant that encourage or initiate nasal congesting and cough.

In reference to the selected ingredients, the quantity of thymol 14 provides antimicrobial effects and powerful antioxidant properties for the quantity of antibiotic ointment 13. The quantity of eucalyptus oil 15 provides antiseptic properties to fight against decongestants within the quantity of antibiotic ointment 13. The blend of lemon, clove, cinnamon bark, eucalyptus radiata, and rosemary 16 provides an essential blend that helps to support healthy immune function within the quantity of antibiotic ointment 13. The quantity of lavender oil 17 is incorporated into the quantity of antibiotic ointment 13 in order to provide aromatic properties. The quantity of cedarwood oil 18 provides antibacterial and fungicide properties for the quantity of antibiotic ointment 13. The quantity of tea tree oil 19 provides beneficial properties to prevent of various infectious diseases within the quantity of antibiotic ointment 13 as it functions as an antimicrobial agent. Furthermore, the quantity of tea tree oil 19 shows potent antimicrobial activity against dermatologic conditions. The quantity of peppermint oil 20, which is extracted from peppermint trees, provides antibacterial, cooling, and aromatic properties within the quantity of antibiotic ointment 13. The quantity of rosemary oil 24, which is extracted from Rosmarinus officinalis that is commonly known as rosemary tree, provides variety of medical benefits. For example, rosmarinic acid is a phytochemical of the rosemary oil and provides antioxidant properties within the quantity of antibiotic ointment 13. Camphor is another phytochemical of the rosemary oil and acts as antimicrobial agent within the quantity of antibiotic ointment 13. The quantity of oregano oil 25 is extracted from perennial herd oregano and provides aromatic properties and anti-disease effects with in the quantity of antibiotic ointment 13.

The fastening mechanism 21 that allows the present invention to be secured to the mask 23 is preferably an adhesive layer 22. More specifically, the adhesive layer 22 is superimposed onto a rear surface 3 of the flexible sheet 1 and covered with at least one backing. Before the present invention is adhered onto the mask 23, the backing should be removed, exposing the adhesive layer 22. The adhesive layer 22 is preferably made of an acrylate, including methacrylates and epoxy diacrylate that are also known as vinyl resin. Even though the adhesive layer 22 is the preferred fastening mechanism 21, the present invention can utilize any other types of fasteners that can easily secure the flexible sheet 1 to the mask 23 as the fastening mechanism 21.

The present invention provides an inexpensive solution to effectively purifies breathing air. More specifically, the users can utilize any types of mask 23 depending upon their work requirements or personal preference with the present invention, wherein the mask 23 and the present invention mutually complete the air purification process for breathing air. Once the effectiveness of the present invention is weakened or depleted overtime, which can be pre-defined by the manufactures and printed on the present invention, the users can remove the used antibacterial breathing strip for nasal decongestant and cough suppressant from the mask 23 and replace that with a newer antibacterial breathing strip for nasal decongestant and cough suppressant. As a result, the users can successfully attain air purification process for breathing air through the present invention.

Although the invention has been explained in relation to its preferred embodiment, it is to be understood that many other possible modifications and variations can be made without departing from the spirit and scope of the invention as hereinafter claimed.

What is claimed is:

1. An antibacterial breathing strip for nasal decongestant and cough suppressant comprises:
a flexible sheet;
an enclosing panel;
a plurality of ventilation openings;
a filter pad;
a fastening mechanism;
the plurality of ventilation openings traversing through the enclosing panel;
the filter pad being positioned in between the flexible sheet and the enclosing panel;
the enclosing panel being perimetrically connected onto the flexible sheet; and
the fastening mechanism being coupled onto the flexible sheet, opposite of the enclosing panel.

2. The antibacterial breathing strip for nasal decongestant and cough suppressant as claimed in claim 1 comprises:
the fastening mechanism being an adhesive layer; and
the adhesive layer being superimposed onto a rear surface of the flexible sheet.

3. The antibacterial breathing strip for nasal decongestant and cough suppressant as claimed in claim 1 comprises:
the enclosing panel comprises a proximal edge, a distal edge, a first lateral edge, and a second lateral edge;
the first lateral edge and the second lateral edge being extended in between the proximal edge and the distal edge;
the proximal edge being terminally connected to a top edge of the flexible sheet;
the distal edge being terminally connected to a bottom edge of the flexible sheet; and
the first lateral edge and the second lateral edge being connected to a front surface of the flexible sheet.

4. The antibacterial breathing strip for nasal decongestant and cough suppressant as claimed in claim 1, wherein the flexible sheet is a permeable sheet.

5. The antibacterial breathing strip for nasal decongestant and cough suppressant as claimed in claim 1, wherein the filter pad is enclosed within the flexible sheet and the enclosing panel.

6. The antibacterial breathing strip for nasal decongestant and cough suppressant as claimed in claim 1 comprises:
a quantity of antibiotic ointment; and
the filter pad being saturated with the quantity of antibiotic ointment.

7. The antibacterial breathing strip for nasal decongestant and cough suppressant as claimed in claim 6 comprises:
the quantity of antibiotic ointment comprises ingredient selected from the group consisting of a quantity of thymol, a quantity of eucalyptus oil, a blend of lemon, clove, cinnamon bark, eucalyptus radiata, and rosemary, a quantity of lavender oil, a quantity of cedarwood oil, a quantity of tea tree oil, a quantity of peppermint oil, a quantity of rosemary oil, a quantity of oregano oil, and combinations thereof.

8. The antibacterial breathing strip for nasal decongestant and cough suppressant as claimed in claim 1, wherein the fastening mechanism is attached to a mask.
